(19) United States
(12) Patent Application Publication
(10) Pub. No.: US 2012/0004672 A1
(43) Pub. Date: Jan. 5, 2012

(54) Suture Straightening Device and Method

(76) Inventors: Brandon Giap, San Diego, CA (US); Donn K. Harms, Del Mar, CA (US)

(21) Appl. No.: 13/174,712

(22) Filed: Jun. 30, 2011

Related U.S. Application Data
(60) Provisional application No. 61/360,380, filed on Jun. 30, 2010.

(51) Int. Cl. A61B 17/04

(52) U.S. Cl. 606/148

(57) ABSTRACT

A device for straightening sutures or pre-packaged suture-needle combinations. The device has a body formed of substantially resilient material flexible enough to be penetrated by a pointed end of a needle along with a suture inserted into it or to obtain a substantial gap when a slit is cut or molded into it. The device may be held securely in a position for subsequent grasping by a user for drawing a suture through it or attached to an OEM suture packaging. A means of engagement such as adhesive provides means for temporary or permanent attachment to the suture packaging.

### BACKGROUND OF THE INVENTION

1. Field of the Invention

This application claims priority to U.S. Provisional Application Ser. No. 61/360,380 filed Jun. 30, 2010, and is incorporated in its entirety by reference.

The disclosed device herein relates generally to sterile suturing products employed by medical professionals for suturing lacerations and incisions and internal tissue on humans and animals. More specifically, the disclosed device and method relate to a device employed with a sterile suturing kit employed in surgical procedures which dispenses the suturing material in a straightened condition rather than the coiled condition such material dispenses from conventional circular storage containers.

2. Prior Art

Modern medicine for animals and humans frequently requires that the patient undergo surgical procedures during treatment for illness or other infirmities which requires incisions internally and externally. Additionally, it is common for patients who have injured themselves to require suturing of lacerations and other injuries. As a consequence, the need to suture lacerations and the like from both injuries and surgical procedures, is a day-to-day requirement in most hospitals, surgical centers and with other medical treatment providers.

As a convenience to the surgeon or medical professional performing the surgery, and as necessitated by the strict sterility requirements to avoid patient infection, both the needle and the suture material must at all times remain sterile prior to use. Further, in order to save time and to avoid the need for the surgeon or medical professional to engage the suture material with the suturing needle, conventionally, such needles and suture material are provided with the suture material operatively engaged with the proximate end of the needle to be employed by the surgeon.

As a consequence of the strict sterility requirements, as well as the conventional provision of needles which are preengaged with suture material, the packaging in which a suture-needle combination is provided by manufacturers has become a key component in the safe and efficient execution of the surgical suturing procedure.

Due to the nature of their use, in a crowded surgical room where the user generally wears latex gloves, suture packaging works best if it is compact, maintains the sterility of the suturing contents, and allows for easy removal of long strands of suture material which is already engaged with the needle to be employed. Such packaging and suture and needle combinations are taught in prior art such as U.S. Pat. Nos. 5,129,511; 5,220,769; 5,284,240; 5,341,922 and 5,439,102 which are included herein by reference thereto.

As disclosed in the prior art, and in what has become a conventional manner of packaging and supply for the medical industry, the sterile packaging provides the suture material in a determined length for the surgeon. The length of the suture strand, for convenience, is much longer than the length of the packaging. In order to provide these long suture lengths, it requires that the suture material be wound within or upon a packaging structure in a circular fashion, either upon a suture holder, or a device adapted to maintain the suture material wound prior to opening and use of the suture material and needle within the package.

In current use of such suturing packs, the user or an assistant will open the package housing the sterile suturing material which is pre-engaged with the needle, and remove it. Once the needle-suture combination is removed from the packaging to be employed by the surgeon or medical professional, a vexing problem arises for the surgeon. The suture material, which is frequently monofilament thread engaged to the needle, will maintain a memory of the shape of the holder upon which it has been wound and stored. This memory effect happens with other types of suturing thread be it wound, spunbond, or monofilament.

This curled trailing strand of suturing material engaged to the rear of a needle results in the user holding the needle with the suture material trailing from the proximate end of the needle and deformed by a multitude of curls and twists having been imparted to it from being wound on a holder and stored in the package. The tighter the wind during storage and the longer the suture material has been held wound on a holder, the more such curls and bends are memorized into the suture strand when deployed and hanging from the needle.

While the suture material is perfectly useable in this state, the mess of bunched up suturing thread or monofilament is a distracting nuisance to a surgeon or medical professional who must use it. The act of suturing a wound or laceration or surgical incision is a delicate process which requires years of practice to master. Many different types of suturing techniques and knots are employed by surgeons in closing a wound or incision depending upon the desired outcome of the healed patient. This suturing may occur on the outside of the body, or it can occur in layers below the skin, or in the tight confines of the mouth.

During this process, the surgeon is delicately trying to guide the needle end into and out of a patient's wound using their fingers or forceps. Concurrently, the surgeon is trying to maintain an even line, even tension, and position a determined number of stitches over a given length.

It is during this process that the curls and bends of the trailing suture material become a constant nuisance to the medical professional performing the suturing. The curved and curled suture strand is hard to see and can easily become unintentionally entangled in the individual sutures or stitches being delicately placed by the surgeon. In confined spaces such as the mouth, the nuisance of the curled suture strand is multiplied. It is not an infrequent occurrence where the surgeon is required to actually remove stitches or sutures because the curled trailing suture strand has become caught in a prior stitch or wrapped around something it should not, or has become knotted.

As such, there exists an unmet need for a device and method which will allow surgeons and medical professionals to employ wound suture material from kits and eliminate the noted problems therewith. Such a device should easily straighten the suture material engaged with the needle, while concurrently being compact and easily housed within the sterile packaging. Such a device upon removal from the sterile package should be easily employed with the suturing material also housed in the sterile packaging. Such a device should be easily employed by users, all of whom are conventionally gloved in latex or other surgical gloves which impair their finger dexterity. Such a device and method should at all costs, protect the sterility of the packaged needle and suture material to maintain the sterile field in the operating room and protect the patient from infection. Finally, such a device should provide means to straighten the dispensed suture while avoiding breakage of the delicate monofilament.

### SUMMARY OF THE INVENTION

The disclosed device and method herein provide a remedy to the noted shortcomings of conventional packaged sterile needle and suture kits with wound suturing material housed therein. The device herein described and disclosed provides a straightening component and method for an easily employed means to straighten suture strand trailing from its engagement to the proximal end of a sterile needle.

In use, the device may be employed by itself, mounted to a ring or surgical table. However being small the device may also be easily engaged to the supporting structures of sterile suturing kits so that it may be maintained sterile while housed in sterile packaging with the kit. So housed, it provides for easy employment to straighten curved and curled suturing strands being drawn from the sterile packaging, even with the users wearing surgical gloves. During such use, the device renders the conventional curled suture strand to a substantially straight and curl-less strand ready for suturing. When housed inside the suture kit, the device remains sterile and concurrently protects the sterility of both the needle and the suture material when used, as is required in modern surgical procedures.

The device may be provided in various modes all of which will provide for a straightening of strands of suturing material which has been wound upon a storage component. In particularly preferred modes, the device is configured such that it is attached to the suture and needle holder which is conventionally housed within a wrapper to maintain sterility of the contents.

Upon opening the packaging in a sterile environment the device is easily employed for use. The device positioned on the body of the suture and needle holder employs an elongated soft member having a resilience which allows for a suture or suture-needle combination, to be drawn through it during detachment of the needle and suture material from the housing on which the suture strand was wound up.

The housing on which strands of suture material are wound are conventionally formed of paper, plastic, cardboard, or other material, and feature a planar body and winding guides all of which are housed inside the wrapper. During dismount of the needle and suturing material for use on the patient, the strand of suture material trailing from an attachment to the needle, is drawn through the elongated and preferably pliable body member of the device herein. Pressure from the pliable member itself, or imparted to the pliable member by the user, is communicated to the strand of sliding suture material being drawn through the member. This pressure and the sliding frictional engagement of the strand of suture material through the pliable body, creates a substantially uniform tension which is imparted to the suture strand by the radial pressure on the exterior surface of the strand caused by the member along the suture material being drawn through the member forming the device. This tensioning helps to slightly stretch and realign the material forming the strand of the suture to thereby substantially remove the memorized shape imparted to the strand by winding on the guides of the housing in the container. The entire length of the suture material may be drawn through the elongated member providing a means for straightening and removing any memorized curves or bends. The user is thereby left with a substantially straight strand of suture material hanging from the needle, devoid of coils and twists, all of which remain sterile.

In one mode of the device a force may be exerted about the external circumference of the suture material to impart the tension to the suture material drawn through the member along a pathway formed through the member. This pathway has a diameter substantially equal to or slightly smaller then that of the strand of suture material.

In use in this mode, the suture or needle-suture combination is fed through an aperture communicating with the pathway where the squeezing pressure caused by the smaller diameter of the passage forming the pathway through the pliable or compressible member provides a calculable and determined pressure needed to effectively stretch and straighten out any curves or bends in the suture material. It should be noted that the pressure exerted on the suture is not limited by the equal or smaller passage diameter and the hardness of the material forming the member. By employing a compressible material for the elongated body of the device, such as silicone, additional circumferential pressure may be applied to the strand of suture material by a compression of the member forming the body of the device. This compression force imparts additional pressure against the exterior surface of the suture which may more effectively straighten the suture material of its memorized curves and curls. The compression force may be imparted easily by the gloved fingers and thumb of the user, or using a means for imparting inward bias upon the compressible member such as a spring loaded clip.

In many cases the suture needle employed by the surgeon is curved. As such, the ability to feed the needle through the passageway may be difficult during use in the sterile confines of the operating room by a gloved user. Consequently, the needle, if curved, is best fed through the passage through the compressible member prior to packaging within the sterile wrapper thereby eliminating the need for users to do so.

In another mode of the invention herein, the passage through the elongated member, rather than formed and accessible through apertures in the surface, is instead comprised of an elongated slit along the length of the body of the member. In use, the slit is accessible by a gap communicating to the exterior surface of the member, whereby the needle and/or the suture material may be drawn into the passage formed by the slit.

The slit may taper to a smaller size from the access point to the bottom of the slit forming the passageway through the member. The narrowest part of the slit thereby can be narrower than the diameter of the suture material and impart and determinable pressure to the suture material drawn therethrough. Alternatively, a pinching or compression of the member may also be exerted on the member, to achieve pressure on the suture or increase pressure on the suture material drawn through the compressible member. As with the first mode of the device, employing a defined passage or conduit, drawing the suture through the device having the passageway formed by a slit, will impart the circumferential pressure to the suture material sufficient to cause any and all memorized bends or curves to be straightened.

A particularly preferred mode of the device is as an attached component to existing suture packaging by engagement to housing on which the suture strand is wound. The body of the device is engaged to the card or other structure employed for the winding of the suture material. As noted above, many conventional suture packages wind a predetermined length of a strand of suture material upon a card, or other planar structure having winding guides thereon. The housing or card is placed within a sterile cavity formed by a surrounding package enclosure along with a needle secured to one end. In use, once the package is opened to reveal the sterile cavity, the needled end is grasped and pulled, and pulls along the trailing strand of suture material from the winding guides and the package. It is at this point that the suture material goes into a curved, coiled and twisted state.

It is therefor particularly preferred, to engage the device to the housing or card holding the guides and the wound suture material which is placed inside the sterile cavity of the packaging. The member would of course also be sterile. So positioned within the sterile cavity, the device, in combination with the card or winding component or support, would have the needle pre-threaded through the passageway communicating through the compressible member. In this fashion, once the package is opened to reveal the sterile suture, needle, and device herein, the needle can be grasped and pulled to draw the suture material through the passageway of the member wherein pressure is imparted to the circumference of the suture material. As the needle and suture material are drawn from the package the suture material is concurrently straightened.

Alternatively, the compressible member may be positioned on the card or container or structure on which the strand of suture material is wound and housed inside the sterile cavity of the package. However, a slit may form the passage which allows the user to either draw the suture material through the passageway through the compressible member, or not, if such is not desirable. In either mode, placing the member in engagement on housing for the wound strand of suture such as the card or other component on which the suture material is wound, allows the compressible member to be sterilized and eliminates the need to package the device in a separate sterile package.

As noted, in either mode, where the passageway is formed by a slit, or a conduit, employing compressible material such as silicone for the member body, allows the user to impart more frictional compressive force to the circumferential surface of the suture material drawn therethrough. This can be done by pinching the member between the thumb and a finger, or using an inwardly biased clip engaged to impart force to two sides of the compressible member.

In a third mode of the device herein, which is well adapted for separate employment with packaged suturing kits which do not have the compressible member housed within the packaging, the device may be attached to a clip or other means of engagement of the compressible member to a surgical table or tray. In this mode of the device, a passageway is formed through the compressible member, by either a slit, or conduit. A slit provides an easier engagement for the user, however a conduit may be employed also, or, the member may be provided with no pathway through the compressible body, and the user may push the needle through the compressible body. The strand of suture is drawn through the compressible body thereafter in all modes of formation and employment of the pathway through the compressible body. A compression or means for inward bias of the compressible material of the body may be employed to exert compressive pressure to the compressible body should more pressure on the suture be desired.

In this respect, before explaining at least one object of the invention in detail it is to be understood that the invention is not limited in its application to the details of the construction and to the arrangement of the components set forth in the previous description or illustrated in the drawings. The suture straitening device and method herein is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other methods and systems for exerting a compressive force upon the exterior surface of a suture and for carrying out the several purposes of the present invention. It is important, therefore, that the claims be regarded as including such equivalent construction insofar as they do not depart form the spirit and scope of the present invention.

It is an object of the present invention to provide a sterile means to straighten curved, coiled, or twisted sutures which exit from standard suture packaging.

Another object of the invention is to allow for an economically viable hand held device to straighten strands of suture material in a sterile manner.

Yet another object of the invention is to provide a component for employment in combination with conventionally packaged housings, cards and components on which suture material is wound and stored in a sterile package.

An additional object of the invention is to employ a means of straightening strands of suture material in suture-needle combinations for a plurality of needle types.

These together with other objects and advantages which will become subsequently apparent reside in the details of the construction and method as more fully hereinafter described and claimed, reference being had to the accompanying drawings forming a part thereof, wherein like numerals refer to like parts.

### BRIEF DESCRIPTION OF THE FIGURES

FIG. 1 depicts a perspective view of one mode of the device with a centrally located passage accessible through apertures in the body sidewall and running the length of the body formed of compressible material.

FIG. 1a shows a side view of the device described in FIG. 1 showing the conduit-like internal pathway in dotted line.

FIG. 1b shows a front view of the device of FIG. 1 showing an exaggerated view of aperture providing access for suture strands to the pathway.

FIG. 2 depicts a perspective view of another mode of the device having a slot communicating through the endwalls and with a side edge and running along the length of the compressible body.

FIG. 2a shows a side view of the device described in FIG. 2.

FIG. 2b shows a front view of the device described in FIG. 2 and depicting the decreasing size of the slot to the narrowest point forming the pathway through the body.

FIG. 3 depicts a top view of the device as positioned on a conventional suture package which is housed in a sterile package showing the winding of the strand of suture through a cut away.

FIG. 4 shows a perspective view of the device as positioned on suture housing showing a conventional suture strand winding card which is housed within a sterile package.

FIG. 5 shows a needle being removed from the suture winding housing or card prior to engagement of the strand with the device if the slit mode of the pathway is employed.

FIG. 6 depicts the placement of the suture into the pathway formed by a slit using a living hinge formed on one side of the body to slightly open the slit after the conventional sterile package (not shown) has been removed.

FIG. 7 shows a mode of the device engaged upon the strand housing or winding component or support depicted as a card, and having the needle pre-threaded through the passage in the body of the device of FIG. 1 or FIG. 2.

FIG. 8 depicts a user imparting compression to the compressible body which is engaged to the suture strand winding card or component by compressing it between a thumb and finger.

FIG. 9 depicts a typical example of the curls, coils, and twists, imparted to conventional strands of suture material from being wound upon cards or winding components and housed in a sterile package.

FIG. 10 depicts the result of the process of employing the device herein to exert force to the exterior surface of the suture strand to straighten the strand after being drawn through the body of the device.

FIG. 11 shows one preferred mode of the device engaged to a suture strand winding component or support or card as a unit, and housed within a sterile cavity formed by a package.

FIG. 11a depicts a sliced through FIG. 11 along line 11a and showing the suture strand wound upon guides on the card and a slot providing access to the pathway through the body.

FIG. 12 shows another preferred mode of the device engaged to a suture winding component or support or card as a unit, and with the needle and strand of suture pre-threaded though a conduit forming the pathway through the compressible body.

FIG. 12a depicts a slice through FIG. 12 along line 12a and showing the suture strand wound upon the winding component or support and the aperture providing access to the conduit forming the pathway through the compressible body.

FIG. 13 depicts another preferred mode of the device wherein a second body is engaged to the suture winding component to impart material such as antibiotics to the exterior of the suture after it is communicated through the compressible body.

FIG. 14 is a sectional view along line 14 showing a slot forming the pathway through an absorbent body which imparts material such as an antibiotic to the suture.

FIG. 15 is a sectional view of FIG. 13 along line 15 showing the suture wound on the winding component and a conduit forming the pathway through the compressible body.

FIG. 16 depicts another mode of the device that may be employed separate from an engagement to the suture strand winding component by the employment of an attaching clip and an inwardly biasing clip surrounding the compressible body.

FIG. 17 depicts a compressible body formed of two halves with a first half compressible to provide a compression force to suture material drawn through, and a second half being absorbent and thereby imparting liquid to the suture drawn therethrough.

### DETAILED DESCRIPTION OF PREFERRED EMBODIMENTS OF THE INVENTION

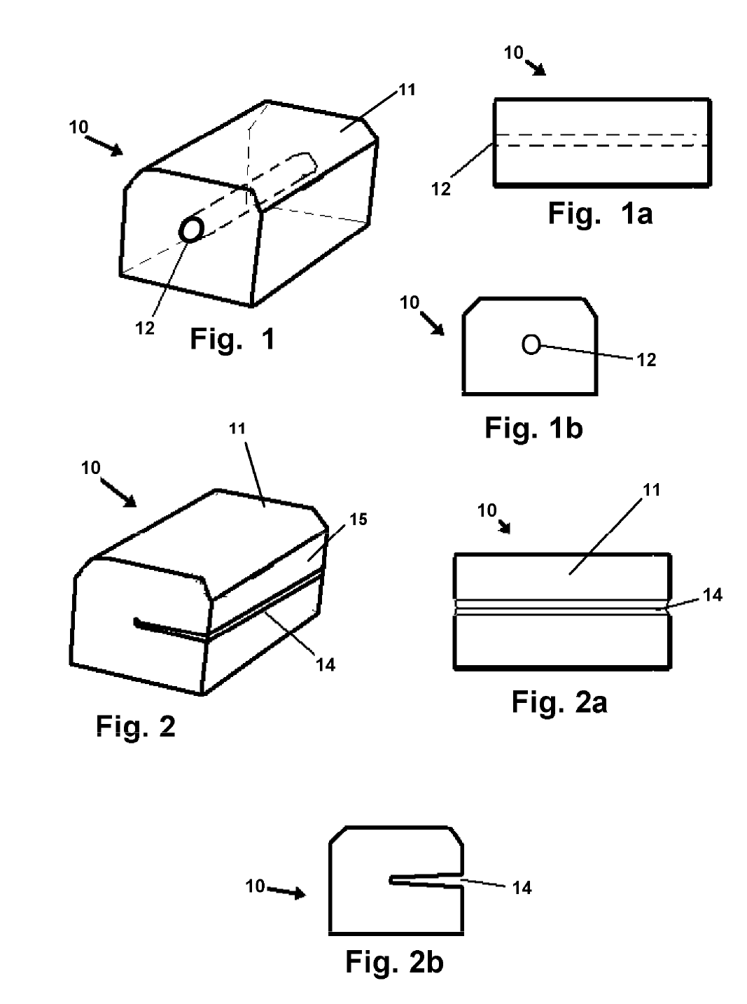

Now referring to drawings in FIGS. 1-17, wherein similar components are identified by like reference numerals, there is seen in FIG. 1 a perspective view of one mode of the device 10 having a body 11 formed of compressible material such as one or a combination of compressible materials from a group of such compressible materials having a shore or durometer between 40-90 including, silicone, polyethylene, natural rubber, and neoprene. All will tolerate the heat of sterilization and have a compressibility to allow the user to pinch the body 11 and deform a centrally disposed passageway 12 to cause it to surround and impart drag or friction upon the outside circumference surface of the strand of suture material causing a slight stretching thereof. The body 11 in all modes of the device 10 has this centrally located passageway 12 which is sized and configured to impart a frictional force to the exterior of a stand of suture 18 drawn therethrough. The passageway 12 of FIG. 1 and 1a, and 1b, is formed much like a conduit communicating through a mid section of the body 11. While it is noted that compressible materials for the body 11 are preferred, a solid or non compressible material may be employed. However, such would also have a much higher incidence of breaking the strand of suture material and is not particularly preferred.

As can be seen in FIG. 1a which is a side view of the body 11 of FIG. 1 the conduit-like passageway communicates through the compressible body 11 to exit apertures 17 which are shown in FIGS. 1 and 1b.

In FIG. 2 a perspective view of another mode of the device 11 having a slot 14 communicating though the compressible body 11. The slot 14 communicates to a side edge 15 of the body 11 and runs the length of the compressible body 11. As shown in FIG. 2a, the slot 14 is accessible at the side edge 15 of the body 11 so that the strand of suture 18 can be engaged or disengaged from the passageway 12 formed by the slot 14. Optionally but preferred, as shown in FIG. 2b the slot 14 may decrease in size until it reaches the passageway 12 running through the compressible body 11.

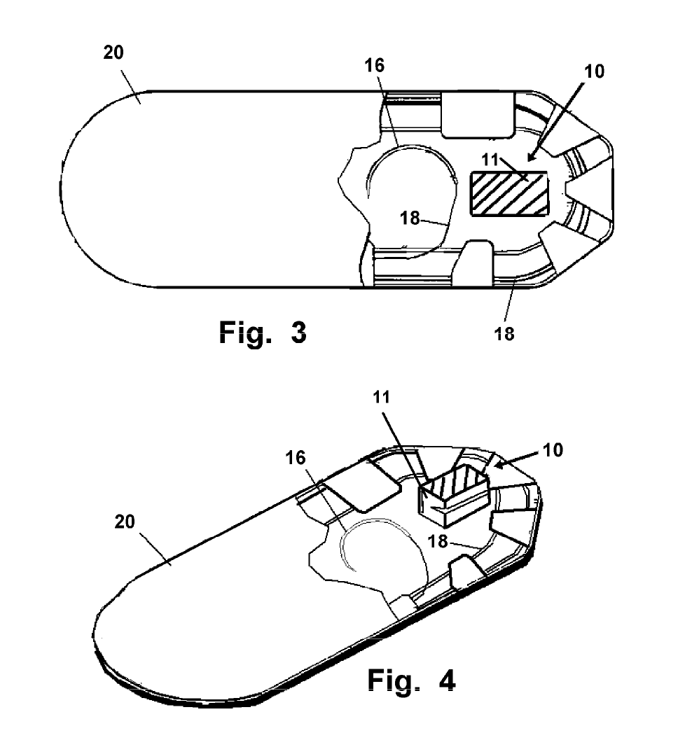

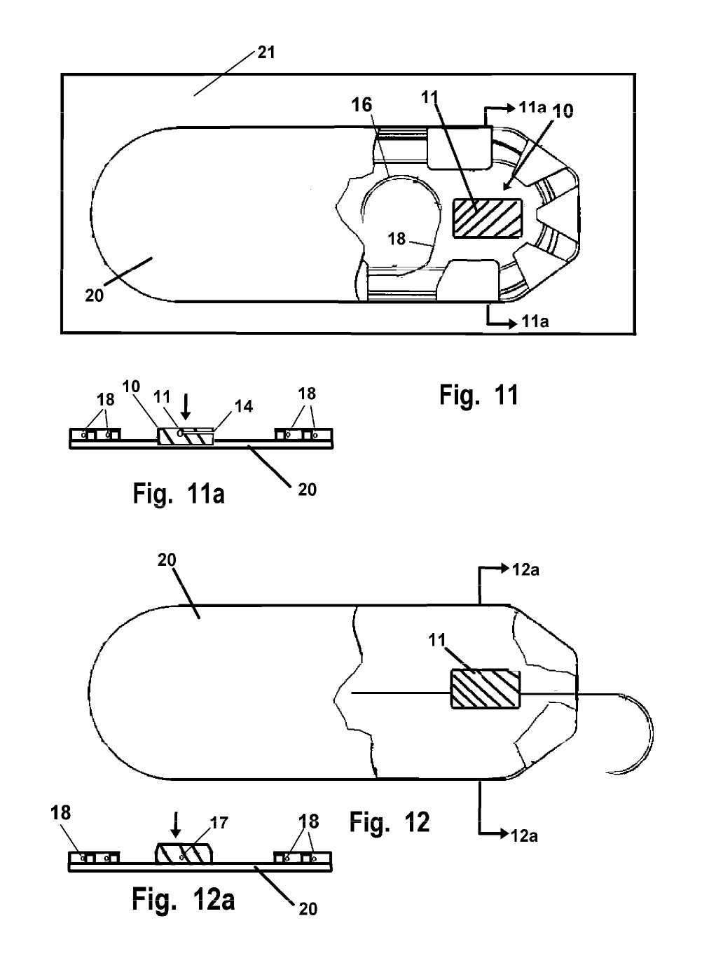

In FIG. 3 is shown a top view of the device 10 as it may be positioned in combination and on a conventional suture strand winding component or support 20 formed of a card or other means for winding the strand of suture 18 for storage within a sterile package 21 shown in FIG. 11. Such suture strand winding components 20 are formed to allow for a winding in a circular pattern of the strand and frequently are formed in planar card-like structure. Positioning the body 11 on the end of the suture strand winding component or support 20 where the needle 16 is pulled to draw the trailing and attached strand of suture 18 allows the strand to be easily drawn through the passageway in the body 11.

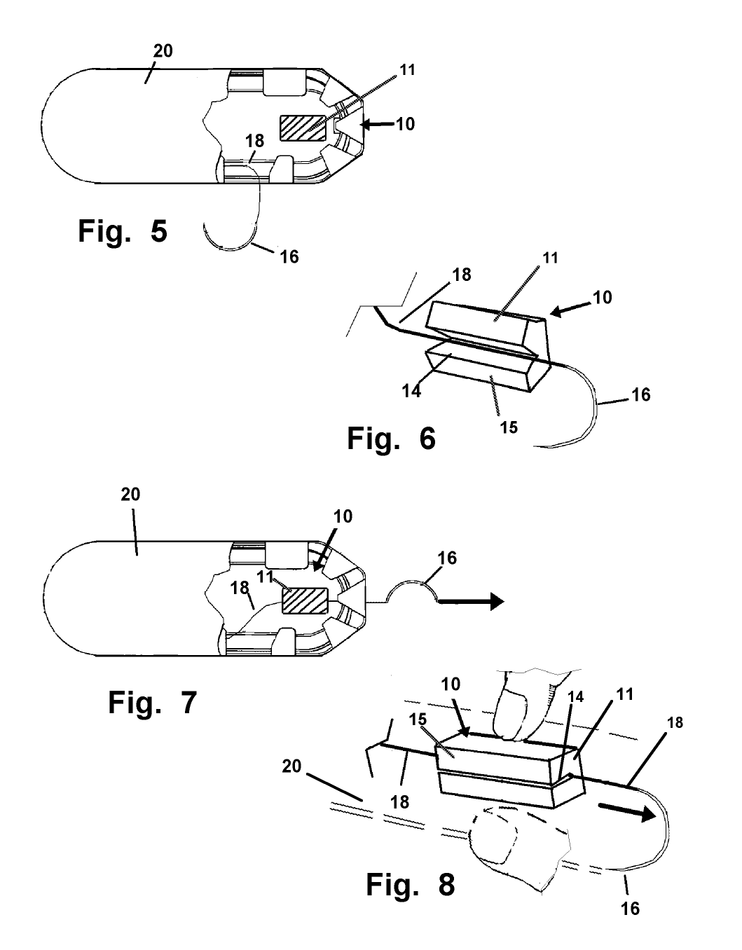

FIG. 4 shows a perspective view of the device 10 where the body 11 is positioned in an engagement on an end of a conventional suture strand winding component or support 20 formed as a card. Depicted is the mode of the body 11 employing the slot 14 which gives the user the option to employ the device 10 or not, depending on preference. As in FIG. 5, if the user does not wish to employ the compressive pressure of the body 11 on the strand of suture 18, they may opt not to leave the strand of suture 18 engaged with the body 11 by removing it if pre-threaded, or not engaging it if not.

The engagement or disengagement of the strand of suture 18 with a body 11 having a slot 14, is shown in FIG. 6. If the strand of suture 18 is pre-threaded through the passageway 12 formed by the slot 14, it can be removed from the slot 14 through the sidewall 15. Alternatively, if the strand of suture 18 is not pre-threaded through the passageway 13 through the body 11, the top portion of the body 11 may be easily rotated to provide easy access to thread the strand of suture 18 thorough the passageway 13 of the compressible body 11.

In FIG. 7 there is shown a mode of the device 10 wherein the body 11 is engaged upon the suture strand winding component 20 depicted as a card. Prior to insertion into a sterile package 21 as in FIG. 11, the needle 16 is pre-threaded through the passageway 12 formed by a conduit like passage through the body 11 of the device 10 of FIG. 1.

All modes of the body 11, either with the slot 14 or with the conduit type of passageway 12, impart a force to the exterior surface of strand of suture material 18 drawn through the passageway 12 running trough the body 11. This can be a set amount of force by forming the passageway 12 to be slightly smaller than the diameter of the strand of suture material 18. Or, as shown in FIG. 8, the force may be increased because the material forming the body 11 is compressible and the user may pinch the body 11 between the finger and thumb to exert extra force to the strand of suture material 18 drawn through the passageway 12. Silicone is particularly preferred for the material forming the body 11 as it will handle the temperatures involved in sterilization and is compressible as noted.

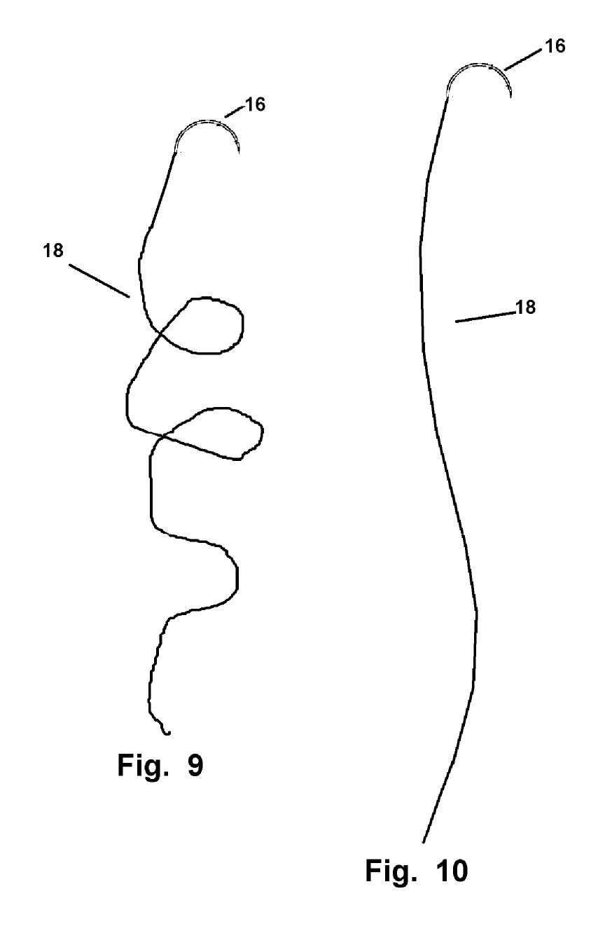

The problem with conventional wound suture kits is best depicted in FIG. 9. This is a typical example of the curls, coils, and twists imparted to conventional strand of suture material 18 when it is wound upon a suture strand winding component 20 such as a card, stored for use. The curls and twists are most annoying to surgeons.

Passage through the passageway 12 of the body 11 subjects the strand of suture material 18 exterior to a compressive force and frictional engagement while drawn through the passageway 12 by a pulling of the user. The force so exerted on the strand of suture material 18, causes a stretching of the strand of suture material 18 which results in a straitening thereof shown in FIG. 10. Employment of material for the body 11 that is compressible provides the ability to pinch the body 11 during a drawing, but limit the amount of force exerted onto the strand of suture material 18 as the body material compresses to absorb some of the pinching force. The durometer of the body material may be changed to exert more or less maximum force as can the material itself. The material itself forming the body 11 can have more or less of a frictional engagement with the strand of suture material 18 during compression. Currently, the body is best formed of one or a combination of compressible materials from a group of such compressible materials having a shore or durometer between 40-90 including, silicone, polyethylene, natural rubber, and neoprene. Experimentation has found that medical grade silicone performs very well and is currently preferred.

FIG. 10 depicts the result of the process of employing the device herein to exert force to the exterior surface of the strand of suture to straighten the strand after being drawn through the body of the device herein.

In FIG. 11 is shown the device 10 provided as a unit adapted to hold the strand of suture material 18 wound on a card like winding component 20. A package 21 provides a means to maintain the device 10 within a sterile cavity until use. When formed as a unit with the device 10 mounted upon the winding component 20 if a slot 14 is employed as in FIG. 11a, the strand of suture material 18 need not be pre-threaded through the passageway 12, but it is preferred to save time for the user.

FIG. 11a depicts a sliced through FIG. 11 along line 11a and showing the strand of suture wound upon the card and a slot providing access to the pathway through the body.

A second mode of the device 10 formed as a unit adapted to hold a strand of suture material 18 wound on a card like winding component 20 is shown in FIG. 12. In this mode, a conduit forms the passageway 12, through the body 11 and pre-threading of the strand of suture material 18 as in FIG. 12 is preferred.

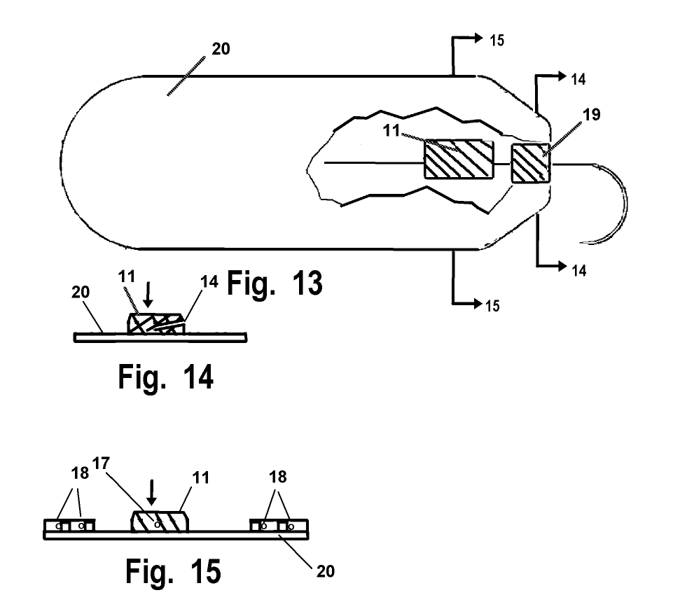

The device 10 may also provide a second body 19 substantially inline with the body 11 through which the passageway 12 is formed as shown in FIG. 13. Formed as a unit much like FIGS. 11 and 12, this mode of the device provides the user with the ability to impart material or liquid such as a lubricant or medicine to the straightened strand of suture material 18 exiting the body 11. The second body 19 best employs a slot 14 as in FIG. 14, for access to the passageway 12 to thereby allow the user the option to pass through the second body 19, after exiting the body 11 as in FIG. 15.

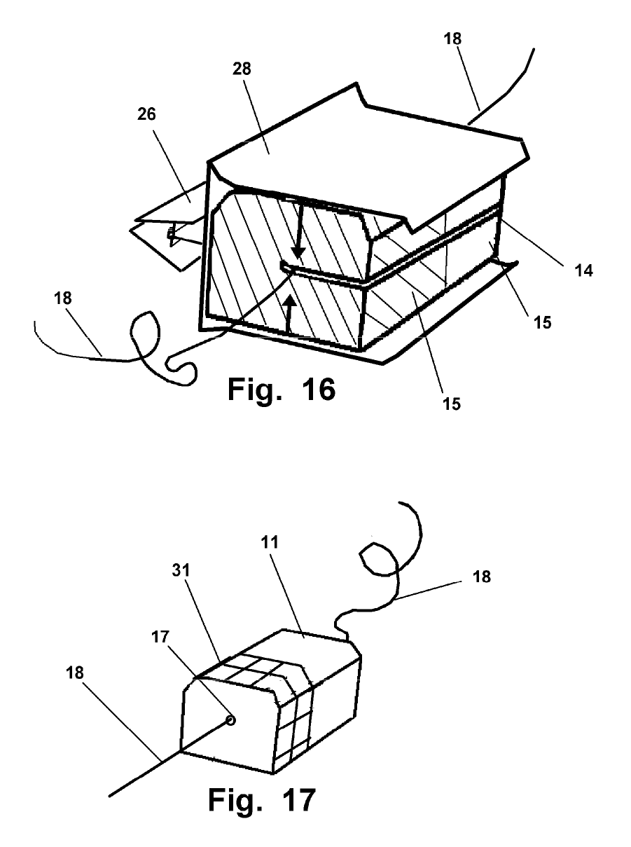

Finally, a stand alone mode of the device 10 is shown in FIG. 16. This mode of the device 10 operates the same as the mode engaged to suture strand winding cards, but may be employed with suture kits not equipped with the device 10 herein. This mode shown in FIG. 16, employs an attaching clip 26 which is engaged to and supports an inwardly biasing body clip 28 surrounding the compressible body 11. The passageway 12 through the body 11 imparts compressive force and frictional engagement to the strand of suture material 18 which can be increased by using a body clip 28 with more inward bias. If application of material to the strand of suture material 18 is desired, the body 11 may have an absorbent half 31 made of sponge or other material which may be soaked with lubricant, or antibiotics and thereby coat the straightened strand of suture material 18 exiting the body 11.

While all of the fundamental characteristics and features of the invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the spirit or scope of the invention. Consequently, all such modifications and variations and substitutions are included within the scope of the invention as defined by the following claims.

What is claimed is:

1. A straightening apparatus for a strand of suture comprising:
a resilient body, said body having a first end and second end;
a passage formed through said body between said first end and said second end;
said passaged sized to exert a frictional engagement upon an exterior surface of said strand of suture, from a proximal end of said strand to a distal end of said strand, during a pulling of said strand through said body from an entrance at said first end to an exit of said strand at said second end; and
said frictional engagement providing means to impart a straightening of said strand exiting said body at said second end.

2. An improved sterile suturing kit wherein a needle engaged to a proximal end of a strand of suture and said strand of suture is wound in a substantially circular fashion upon a support which is housed in a sterile cavity formed by a package, and where said strand of suture when removed from said package is has an irregular shape such as curls or coils, the improvement comprising:
a resilient body, said body having a first end and second end;
a passage formed through said body between said first end and said second end;
said body being substantially sterile and positioned within said sterile cavity;
said passage sized to exert a frictional engagement upon an exterior surface of said strand of suture, from said proximal end of said strand to said distal end of said strand, during a pulling of said strand through said passage between said first end and said second end; and
said frictional engagement providing means to impart a straightening of said strand exiting said body at said second end; whereby said strand is substantially straightened and said irregular shape substantially removed.

3. The improved sterile suturing kit of claim 2 additionally comprising:
said passage formed by a slot formed through a first sidewall of said body extending between said first and second ends; and
said slot proving means to engage said strand to a communication through said body.

4. The improved sterile suturing kit of claim 3 additionally comprising:
said passage formed by a conduit communicating through said body to apertures at said first end and second end; and
said passage having a diameter substantially equal to or slightly smaller than a diameter of said strand.

5. The improved sterile suturing kit of claim 2 additionally comprising:
said body engaged to said support.

6. The improved sterile suturing kit of claim 3 additionally comprising:
said body engaged to said support.

7. The improved sterile suturing kit of claim 4 additionally comprising:
said body engaged to said support.

8. The improved sterile suturing kit of claim 5 additionally comprising:
said strand being preengaged through said passage whereby upon an opening of said package, said strand is drawn to a communication through said body by a pulling of said needle.

9. The improved sterile suturing kit of claim 6 additionally comprising:
said strand being preengaged through said passage whereby upon an opening of said package, said strand is drawn to a communication through said body by a pulling of said needle.

10. The improved sterile suturing kit of claim 7 additionally comprising:
said strand being preengaged through said passage whereby upon an opening of said package, said strand is drawn to a communication through said body by a pulling of said needle.

11. The improved sterile suturing kit of claim 8 additionally comprising:
said resilient body having a durometer allowing for a compression between a thumb and finger of a user; and
said compression providing means for increasing said frictional engagement to provide an increase in said straightening of said strand.

12. The improved sterile suturing kit of claim 9 additionally comprising:
said resilient body having a durometer allowing for a compression between a thumb and finger of a user; and
said compression providing means for increasing said frictional engagement to provide an increase in said straightening of said strand.

13. The improved sterile suturing kit of claim 10 additionally comprising:
said resilient body having a durometer allowing for a compression between a thumb and finger of a user; and
said compression providing means for increasing said frictional engagement to provide an increase in said straightening of said strand.

14. The straightening apparatus of claim 1, additionally comprising:
said passage formed by a slot formed through a first sidewall of said body extending between said first and second ends;
said slot proving means to engage said strand to a communication through said body;
a second sidewall opposite said first sidewall providing a hinged engagement of two portions of said body on opposite sides of said slot; and
said hinged engagement providing means to enlarge said slot in said first sidewall temporarily for an insertion of said strand therein.

15. The straightening apparatus of claim 14, additionally comprising:
said resilient body having a durometer allowing for a compression between a thumb and finger of a user; and
said compression providing means for increasing said frictional engagement to provide an increase in said straightening of said strand.

16. The straightening apparatus of claim 15, additionally comprising:
means to engage said body to a mounted position whereby a user may employ two hands to engage said strand to a communication through said body.

17. The straightening apparatus of claim 16, additionally comprising:
a biasing clamp engaged upon two opposing side surfaces of said body;
an open side of said clamp providing access to said slot in said first sidewall; and
said biasing clamp exerting a compressive force to said body to increase said frictional engagement.

18. The straightening apparatus of claim 1, additionally comprising:
said passage formed by a conduit communicating through said body to apertures at said first end and second end; and
said passage having a diameter substantially equal to or slightly smaller than a diameter of said strand.

19. The straightening apparatus of claim 1, additionally comprising:
said passage formed by a pushing said needle through said body to form said conduit communicating through said body whereby said body is provided in solid form and said passage formed by said user.
